(19) United States
(12) Patent Application Publication
(10) Pub. No.: US 2003/0021763 A1
(43) Pub. Date: Jan. 30, 2003

(54) Method for the treatment and/or prophylaxis of diseases caused by IL-12

(75) Inventors: Tieno Germann, Aachen (DE); Stefanie Frosch, Aachen (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH.

(21) Appl. No.: 10/156,771

(22) Filed: May 29, 2002

Related U.S. Application Data
(63) Continuation of application No. PCT/EP00/11179, filed on Nov. 11, 2000.

(30) Foreign Application Priority Data
Nov. 29, 1999 (DE) 199 57 342.5

(51) Int. Cl. A61K 38/19; A61K 31/454

(52) U.S. Cl. 424/85.1; 514/323

(57) ABSTRACT

A method for the treatment or prophylaxis of a disease caused by the production of IL-12, comprising administering to a subject in need thereof a compound selected from the group consisting of thalidomide, α-methyl thalidomide (EM 978), and 3-(1-oxo-1,3-dihydro-isoindol-2-yl)-piperidine-2,6-dione (EM 12), simultaneously with an anti-inflammatory cytokine in an amount effective for inhibiting IL-12 production. Also disclosed is a method for inhibiting IL-12 production in a cell that is capable of producing IL-12.

### CROSS REFERENCE TO RELATED APPLICATIONS

The present application is a continuation of international patent application no. PCT/EP00/11179, filed Nov. 11, 2000, designating the United States of America, the entire disclosure of which is incorporated herein by reference. Priority is claimed based on Federal Republic of Germany patent application no. DE 199 57 342.5, filed Nov. 29, 1999.

### BACKGROUND OF THE INVENTION

The present invention relates to a combination therapy for the treatment of diseases caused by the formation of the pro-inflammatory cytokine IL-12.

IL-12 is a heterodimeric molecule consisting of covalently bonded p35 and p40 chains. The molecule is formed by antigen presenting cells (monocytes/macrophages, dendritic cells, B lymphocytes). The formation of IL-12 by monocytes/macrophages is triggered either by various microbial products, such as lipopolysaccharide (LPS), lipopeptides, bacterial DNA, or in interaction with activated T lymphocytes (Trinchieri 1995. Ann. Rev. Immunol. 13: 251). IL-12 is of central immunoregulating importance and is responsible for the development of inflammation-promoting TH1 reactivities. The presence of a TH1 immune reaction against self antigens leads to the occurrence of serious diseases.

The importance of pro-inflammatory cytokines such as IL-12 in the development and progression of inflammations or autoimmune diseases is clearly documented on the basis of numerous animal experiments and first clinical trials. The pathophysiological importance of IL-12 can be seen in various animal models of diseases such as rheumatoid arthritis, multiple sclerosis, diabetes mellitus and inflammatory diseases of the intestine, the skin and the mucous membranes (Trembleau et al. 1995. Immunol. Today 16: 383; Müller et al. 1995. J. Immunol. 155: 4661; Neurath et al. 1995. J. Exp. Med. 182: 1281; Segal et al. 1998. J. Exp. Med. 187: 537; Powrie et al. 1995. Immunity 3: 171; Rudolphi et al. 1996. Eur. J. Immunol. 26: 1156; Bregenholt et al. 1998. Eur. J. Immunol. 28: 379). By the administration of IL-12 it was possible to trigger the disease in question, or after the neutralization of endogenous IL-12 a diminished progression of the disease was observed until the animals were healed. The use of antibodies against IL-12 in humans is still to come.

It can be stated in summary that an excess of IL-12 determines the pathophysiology of a large number of inflammatory diseases. Attempts at normalizing the IL-12 level therefore have great therapeutic potential.

In addition, IL-12 is also involved in the regulation of the survival of cells. Uncontrolled cell growth is regulated inter alia by apoptosis (programmed cell death). With reference to T lymphocytes it has been found that IL-12 possesses anti-apoptotic activity and promotes the survival of T-cells (Clerici et al. 1994. Proc. Natl. Acad. Sci. USA 91: 11811; Estaquier et al. 1995. J. Exp. Med. 182: 1759). A local overproduction of IL-12 may, therefore, contribute towards the survival of tumor cells.

IL-10 is a cytokine which was originally described as “cytokine synthesis inhibitory factor” (Fiorentino et al. 1989. J. Exp. Med. 170: 2081). That means that IL-10 inhibits the synthesis of the pro-inflammatory monokines TNFα, IL-1, IL-6, IL-8, IL-12 and GM-CSF by human and murine monocytes/macrophages (Fiorentino et al. 1991. J. Immunol. 146: 3444; De Waal Malefyt et al. 1991. J. Exp. Med. 174: 1209). In addition, that leads indirectly to an inhibition of the synthesis of IFN-γ by TH1 lymphocytes. IL-10 is produced by various cells, including a particular T lymphocyte population, B lymphocytes as well as monocytes/macrophages themselves. Interestingly, the formation of IL-10 by monocytes/macrophages occurs with a slight time delay as compared with the synthesis of the pro-inflammatory cytokines. More recent studies show that the pro-inflammatory cytokines TNFα and IL-12 themselves induce the synthesis of synthesis of IL-10 by monocytes/macrophages (van der Poll et al. 1994. J. Exp. Med. 180: 1985; Platzer et al. 1995. Int. Immunol. 7/4: 517) or T-cells (Meyaard et al. 1996. J. Immunol. 156: 2776). The importance of IL-10 in the regulation of mucosal inflammations has been thoroughly investigated in various animal models of inflammatory intestinal disorders. Mice deficient in IL-10 develop serious intestinal inflammation, comparable to that of Crohn's disease (Kuhn et al. 1993. Cell 75: 263). The clinical manifestation was improved by administration of IL-10 to diseased mice or rabbits (Powrie et al. 1994. Immunity 1: 553; Grool et al. 1996. Gastroenterology 110: A918). Moreover, in first clinical trials on patients with ulcerative colitis, the mucosal inflammation regressed following local administration of IL-10 (Schreiber et al. 1995. Gastroenterology 108: 1434; Van Deventer et al. 1997. Gastroenterology 113: 383). The development of inflammatory skin diseases in mice can also be prevented by IL-10 (Enk et al. 1994. J. Exp. Med. 179: 1397). Furthermore, IL-10 is therapeutically effective in skin diseases (psoriasis) in humans (Asadullah et al. 1998. J. Clin. Invest. 101: 783).

In addition to IL-10, a number of other cytokines, such as TGFβ (D'Andrea et al. 1995. J. Exp. Med. 181: 537) and also IL-11 (Leng and Elias. 1997. J. Immunol. 159: 2161) and IFN α/β (Cousens et al. 1997. Proc. Natl. Acad. Sci. USA 94: 634), are capable of inhibiting the formation of IL-12 in macrophages. By way of the inhibition of IL-12, IL-11 imparts protection against tissue inflammations and moderates allergen-induced colitis in rats (Pfeiffer and Qiu. 1995. Gastroenterology 108: A893). Moreover, IL-11 is approved by the FDA for the treatment of thrombocytopenia induced by chemotherapy. The systemic administration of the anti-inflammatory cytokine TGFβ has therapeutic potential in animal models of autoimmune diseases such as experimental allergic encephalomyelitis (Racke et al. 1991. J. Immunol. 146: 3012). α- and β-interferons are already used for the therapy of multiple sclerosis.

The immunomodulatory properties of thalidomide are already being used therapeutically for a number of syndromes such as erythema nodosum leprosum (Sampaio et al. 1993. J. Infect. Dis. 168: 408), cutaneous systemic lupus erythematosus (Atra and Sato 1993. Clin. Exp. Rheumatol. 11: 487), Behcet disease (Hamuryudan et al. 1998. Ann. Intern. Med. 128: 443) and stomatitis aphthosa (Grinspan et al. 1989. Am. Acad. Dermatol. 20: 1060). Although the underlying mechanism of action has not yet been explained, it has been adequately demonstrated that thalidomide is capable of inhibiting the proinflammatory cytokines TNFα (Sampaio et al. 1991. J. Exp. Med. 173: 699) and especially IL-12 (Moller et al. 1997. J. Immunol. 159: 5157), which cytokines, as mentioned above, make a substantial contribution to the pathogenesis of autoimmune diseases and inflammatory reactions.

It is known to use thalidomide in combination with other inflammation-inhibiting, especially steroidal or non-steroidal, active ingredients for the therapy of rheumatoid arthritis (WO 95/04553) and the inhibition of angiogenesis (WO 98/19649).

### SUMMARY OF THE INVENTION

This invention relates to a method for the treatment or prophylaxis of a disease caused by the production of IL-12. In a preferred embodiment, the method of the invention comprises administering to a subject in need thereof a compound selected from the group consisting of thalidomide, α-methyl thalidomide (EM 978), and 3-(1-oxo-1,3-dihydro-isoindol-2-yl)-piperidine-2,6-dione (EM 12), simultaneously with an anti-inflammatory cytokine in an amount effective for inhibiting IL-12 production. A preferred is thalidomide or α-methyl thalidomide (EM 978). Preferred anti-inflammatory cytokine for the present invention is a cytokine selected from the group consisting of IL-10, IL-11, TGFβ, α-interferon, and β-interferon. Preferably, according to the instant method, the compound and the anti-inflammatory cytokine are each administered in a dosage to achieve in the subject a serum concentration ranging from one tenth to 100 times the respective EC₅₀.

In another embodiment, this invention relates to a method for inhibiting IL-12 production in a cell that is capable of producing IL-12. The method comprises exposing the cell simultaneously to an anti-inflammatory cytokine and a compound selected from the group consisting of thalidomide, α-methyl thalidomide (EM 978) and 3-(1-oxo-1,3-dihydro-isoindol-2-yl)-piperidine-2,6-dione (EM 12), in an amount effective for inhibiting IL-12 production.

### DETAILED DESCRIPTION OF THE INVENTION

Surprisingly, it has now been found that thalidomide, its α-methyl compound EM 978 or EM 12 (See Table 1 below) in combination with an anti-inflammatory cytokine, preferably IL-10, IL-11, TGFβ, α- or β-interferon, synergistically inhibits IL-12 production.

Accordingly, the invention provides a method for the treatment and/or prophylaxis of diseases caused by the formation of the pro-inflammatory cytokine IL-12, wherein one of the above-mentioned thalidomide compounds and an anti-inflammatory cytokine, preferably one of those mentioned above, are administered simultaneously according to the invention.

In addition to thalidomide, which is preferred, α-methyl thalidomide (EM 978) is also especially suitable as the thalidomide compound for the combination therapy.

In the case of the simultaneous combined administration of the active ingredients, the anti-inflammatory cytokine advantageously is administered by the parenteral route, that is to say the subcutaneous, intramuscular or intravenous route.

The thalidomide compound can be administered by the oral, rectal, ophthalmic (intravitreal, intracameral), nasal, topical (including buccal or sublingual), vaginal or parenteral (including subcutaneous, intramuscular, intravenous, intradermal, intratracheal or epidural) route.

The amounts of the active ingredients to be used and the choice of adjuvants, such as carriers, fillers, solvents, diluents, colorants and/or binders, with which the active ingredient in question is processed to a particular form of administration, depend on the nature of the administration.

For oral administration there are suitable preparations in the form of tablets, chewing tablets, dragees, capsules, granules, drops, juices or syrups, and for parenteral and topical administration and for administration by inhalation there are suitable solutions, suspensions, readily reconstitutable dry preparations, and also sprays. Examples of suitable percutaneous forms of administration to be used according to the invention are thalidomide compounds in a depot in dissolved form, in a carrier film or in a plaster, optionally with the addition of agents promoting penetration of the skin. The compounds can be released in a delayed manner from preparations that are administrable orally or percutaneously. Ophthalmic forms of administration include drops, ointments and gels.

The total amount of active ingredient to be administered to patients varies depending on the weight of the patient, on the nature of the administration, on the indication and on the severity of the disease. From 1 to 150 mg/kg of a combination according to the invention are usually administered.

The method of treatment according to the invention is suitable for the therapy and/or prophylaxis of diseases in which excessive IL-12 production is held to be responsible for the pathogenesis (inter alia diseases of the intestine, of the skin, of the mucous membranes, of the vessels, and also autoimmune diseases). The combined administration of thalidomide/thalidomide analogs and anti-inflammatory cytokine is also suitable for the therapy of haematological diseases and further oncological diseases. The synergistic action of thalidomide/thalidomide analogs with cytokines such as IL-10 is distinguished at the optimum dose by almost complete inhibition of IL-12 production in LPS-activated monocytes. Even when the combined dose is sub-optimal, higher degrees of inhibition are achieved than with the respective individual doses. The concentrations of the various inhibitors necessary for combined use are therefore lower than the required individual concentrations, and far fewer side-effects of the individual inhibitors are therefore to be expected. In addition, owing to the synergistic action which is observable over a wide range of doses, it is possible to determine a suitable therapeutically effective dose according to the severity of the symptoms.

The active ingredients are used for the treatment of the mentioned diseases in a range of doses which achieves a serum concentration in the range of about 0.1 times the respective EC₅₀ dosage to about 100 times the EC₅₀. EC₅₀ values for thalidomide/thalidomide analogs are from 50 to 100 ng/ml, and the EC₅₀ value for IL-10 is 50 pg/ml.

The diseases of the above-mentioned type include inter alia inflammations of the skin (e.g. atopic dermatitis, psoriasis, eczema, sclerodermia), inflammations of the airways (e.g. bronchitis, pneumonia, bronchial asthma, ARDS (adult respiratory distress syndrome), sarcoidosis, silicosis/fibrosis), inflammations of the gastrointestinal tract (e.g. gastroduodenal ulcers, Crohn's disease, ulcerative colitis), also diseases such as hepatitis, pancreatitis, appendicitis, peritonitis, nephritis, aphthosis, conjunctivitis, keratitis, uveitis, retinopathy, rhinitis.

The autoimmune diseases include, for example, diseases of the arthritic type (e.g. rheumatoid arthritis, HLA-B27 associated diseases), also multiple sclerosis, youthful diabetes or lupus erythematosus.

Further indications are sepsis, bacterial meningitis, chronic bacterial and chronic viral infections (e.g. HIV/AIDS, hepatitis), cachexia, transplant rejection reactions, graft-versus-host reactions, atherosclerosis, and also the reperfusion syndrome/heart failure and tumor diseases.

The syndromes to be inhibited by the combined administration of thalidomide/thalidomide analogues and anti-inflammatory cytokine also include haematological diseases such as multiple myeloma and leukemias, as well as other oncological diseases such a glioblastoma, prostate carcinoma and mammary carcinoma.

### EXAMPLES

TABLE 1
Immunomodulators used

| Substance | Name |
|---|---|
| Thalidomide | 2-(2,6-dioxo-piperidin-3-yl)-isoindole-1,3-dione |
| EM 978 | α-methyl thalidomide |
| EM 12 | 3-(1-oxo-1,3-dihydro-isoindol-2-yl)-piperidine-2,6-dione |

Stimulation of Human Monocytes with Lipopolysaccharide to Secrete IL-12

Human monocytes were isolated from peripheral blood mononuclear cells (PBMC) which had been obtained from heparinized full blood by a Ficoll density gradient centrifugation. To that end, the PBMC were incubated with a monoclonal antibody directed against the monocyte-specific surface molecule CD14 and to which superparamagnetic microbeads (Miltenyi Biotech, Bergisch Gladbach) were coupled. For the positive selection of the labelled monocytes from the PBMC cell mixture, the total cell suspension was applied to a column having a ferromagnetic carrier matrix, and the column was placed in a magnetic field. The cells charged with microbeads were thus bonded to the carrier matrix, while unlabelled cells passed through the column and were discarded. After removing the matrix from the magnetic field, the antibody-charged cells were eluted by flushing the now demagnetised column with buffer. The purity of the resulting CD14-positive monocyte population was approximately from 95 to 98%. The monocytes were incubated for one hour at 37° C. and 5% CO₂ in a density of 10⁶cells/ml culture medium (RPMI, supplemented with 10% foetal calf serum) with the test substances dissolved in DMSO. 20 μg/ml of LPS from E. coli were then added. After 24 hours, cell-free culture supernatants were collected and tested for their IL-12 content.

The concentration of IL-12 in the cell culture supernatants was determined by sandwich ELISA's using two anti-IL-12 monoclonal antibodies (Biosource Europe, Fleurus, Belgium). A reference standard curve with human IL-12 was included. The detection limit of the IL-12 ELISA was 10 pg/ml.

TABLE 2
Effect of thalidomide and interleukin-10 on the IL-12 production of
LPS-activated monocytes

| | | | |
|---|---|---|---|
| Thalidomide | Thalidomide | Thalidomide | Thalidomide |
| 0 μg/ml | 5 μg/ml | 0.5 μg/ml | 0.05 μg/ml |

| | | | | | | | |
|---|---|---|---|---|---|---|---|
| pg/ml* | % Inh.** | pg/ml | % Inh. | pg/ml | % Inh. | pg/ml | % Inh. |

| | | | | | | | | |
|---|---|---|---|---|---|---|---|---|
| IL-10 | 6084 | 0 | 1481 | 76 | 2037 | 67 | 3369 | 45 |
| 0 pg/ml | | | | | | | | |
| IL-10 | 2534 | 58 | 447 | 93 | 618 | 90 | 809 | 87 |
| 50 pg/ml | | | | | | | | |
| IL-10 | 5637 | 7 | 1057 | 83 | 1147 | 81 | 1958 | 68 |
| 5 pg/ml | | | | | | | | |

*IL-12 production
**% Inh. = % inhibition of the IL-12 production of the control without inhibitors

TABLE 3
Effect of EM 978 and interleukin-10 on the IL-12 production of LPS-
activated monocytes

| | EM 978 | | EM 978 | | EM 978 | | EM 978 | |
|---|---|---|---|---|---|---|---|---|
| | 0 μg/ml | | 0.5 μg/ml | | 0.05 μg/ml | | 0.005 μg/ml | |
| | pg/ml* | % Inh.** | pg/ml | % Inh. | pg/ml | % Inh. | pg/ml | % Inh. |
| IL-10 | 4890 | 0 | 439 | 91 | 3161 | 35 | 4571 | 7 |
| 0 pg/ml | | | | | | | | |
| IL-10 | 2538 | 47 | 191 | 96 | 1555 | 68 | 2302 | 53 |
| 50 pg/ml | | | | | | | | |
| IL-10 | 4393 | 10 | 272 | 94 | 1897 | 61 | 2922 | 40 |
| 5 pg/ml | | | | | | | | |

TABLE 4
Effect of EM 12 and interleukin-10 on the IL-12 production of LPS-
activated monocytes

| | | | |
|---|---|---|---|
| EM 12 | EM 12 | EM 12 | EM 12 |
| 0 μg/ml | 5 μg/ml | 0.5 μg/ml | 0.05 μg/ml |

| | pg/ml* | % Inh.** | pg/ml | % Inh. | pg/ml | % Inh. | pg/ml | % Inh. |
|---|---|---|---|---|---|---|---|---|
| IL-10 | 6537 | 0 | 2220 | 66 | 2098 | 68 | 3478 | 47 |
| 0 pg/ml | | | | | | | | |
| IL-10 | 1237 | 81 | 546 | 92 | 649 | 90 | 589 | 91 |
| 50 pg/ml | | | | | | | | |
| IL-10 | 4581 | 30 | 1271 | 81 | 1462 | 78 | 2944 | 55 |
| 5 pg/ml | | | | | | | | |

The results shown in Tables 2 to 4 show the dose-dependent inhibitory action of thalidomide, EM 12, EM 978 and also IL-10 on the IL-12 production of LPS-stimulated monocytes. Surprisingly, a markedly increased inhibition can be observed by the simultaneous combined administration of IL-10 and thalidomide/thalidomide analogues. The increase in the inhibition is to be seen with both optimum and sub-optimum concentrations of both classes of inhibitor. An almost complete inhibition of IL-12 can be achieved by a combination of higher thalidomide/thalidomide analog concentration and higher interleukin-10 concentration.

By using the α-methyl compound of thalidomide, EM 978, a concentration that is 10 times lower than in the case of thalidomide or EM 12 can be used in order to achieve the same effects.

By the combined administration of two different classes of immunomodulators, thalidomide/thalidomide analogs, together with anti-inflammatory cytokines such as IL-10, a synergistic enhancement of IL-12 inhibition is achieved on the one hand, and on the other hand a reduction in the required dose of inhibitor is also achieved. A combination therapy thus permits much more efficient inhibition of the inflammation-promoting mediator IL-12. Furthermore, by reducing the individual doses required for the inhibition, a reduction in undesirable side-effects is to be expected.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

All references cited above are expressly incorporated herein by reference.

What is claimed is:

1. A method for the treatment or prophylaxis of a disease caused by the production of IL-12, the method comprising administering to a subject in need thereof a compound selected from the group consisting of thalidomide, α-methyl thalidomide (EM 978), and 3-(1-oxo-1,3-dihydro-isoindol-2-yl)-piperidine-2,6-dione (EM 12), simultaneously with an anti-inflammatory cytokine in an amount effective for inhibiting IL-12 production.

2. A method according to claim 1, wherein the compound is thalidomide or α-methyl thalidomide (EM 978).

3. A method according to claim 1, wherein the anti-inflammatory cytokine is selected from the group consisting of IL-10, IL-11, TGFβ, α-interferon and β-interferon.

4. A method according to claim 1, wherein the compound and the anti-inflammatory cytokine each has an EC₅₀ value, and each is administered in a dosage to achieve in the subject a serum concentration ranging from 0.1 to 100 times the respective EC₅₀.

5. A method according to claim 1, wherein compound is administered orally, rectally, ophthalmically, nasally, topically, vaginally or parenterally, and the anti-inflammatory cytokine is administered parenterally.

6. A method according to claim 5, wherein compound is administered intravitreally, intracamerally, buccally or sublingually, and the anti-inflammatory cytokine is administered subcutaneously, intramuscularly or intravenously.

7. A method according to claim 1, wherein the compound is simultaneously administered with the anti-inflammatory cytokine via the same route.

8. A method according to claim 1, wherein the compound is simultaneously administered with the anti-inflammatory cytokine via a different route.

9. A method according to claim 1, wherein the disease is selected from the group consisting of atopic dermatitis, psoriasis, eczema, sclerodermia, bronchitis, pneumonia, bronchial asthma, adult respiratory distress syndrome (ARDS), sarcoidosis, silicosis, fibrosis, gastroduodenal ulcers, Crohn's disease, ulcerative colitis, hepatitis, pancreatitis, appendicitis, peritonitis, nephritis, aphthosis, conjunctivitis, keratitis, uveitis, retinopathy, rhinitis, rheumatoid arthritis, HLA-B27 associated diseases, multiple sclerosis, youthful diabetes lupus erythematosus, sepsis, bacterial meningitis, HIV infection, AIDS, cachexia, transplant rejection reactions, graft-versus-host reactions, atherosclerosis, reperfusion syndrome, heart failure, myeloma, leukaemia, glioblastoma, prostate carcinoma and mammary carcinoma.

10. A method for inhibiting IL-12 production in a cell capable of producing IL-12, comprising exposing the cell simultaneously to an anti-inflammatory cytokine and a compound selected from the group consisting of thalidomide, α-methyl thalidomide (EM 978) and 3-(1-oxo-1,3-dihydro-isoindol-2-yl)-piperidine-2,6-dione (EM 12), in an amount effective for inhibiting IL-12 production.

11. A method according to claim 10, wherein the compound is thalidomide or α-methyl thalidomide (EM 978).

12. A method according to claim 10, wherein the anti-inflammatory cytokine is selected from the group consisting of IL-10, IL-11, TGFβ, α-interferon and β-interferon.

13. A method according to claim 10, wherein the compound and the anti-inflammatory cytokine each has an EC₅₀ value, and the cell is exposed to each in a concentration ranging from 0.1 to 100 times the respective EC₅₀.
